(19) United States
(12) Patent Application Publication
(10) Pub. No.: US 2004/0053399 A1
(43) Pub. Date: Mar. 18, 2004

(54) Methods and compositions for analyzing polymers using chimeric tags

(76) Inventor: Rudolf Gilmanshin, Waltham, MA (US)

Correspondence Address: Helen C. Lockhart, Wolf, Greenfield & Sacks, P.C., Federal Reserve Plaza, 600 Atlantic Avenue, Boston, MA, 02210, US

(21) Appl. No.: 10/622,076

(22) Filed: Jul. 17, 2003

Related U.S. Application Data
(60) Provisional application No. 60/396,919, filed on Jul. 17, 2002.

(51) Int. Cl. C12Q 1/68; C12N 1/20

(52) U.S. Cl. 435/252.3; 435/6

(57) ABSTRACT

The invention relates to the use of conjugates of nucleic acid tag molecules and nucleic acid binding agents for labeling polymers such as nucleic acid molecules. The nucleic acid binding agents are nucleic acid binding enzymes that bind nucleic acid molecules non-specifically, in some embodiments. The conjugate can be formed by directly or indirectly binding the nucleic acid tag molecules to the nucleic acid binding agents. The invention provides conjugate compositions as well as methods and systems for using the conjugates to label and analyze polymers such as nucleic acid molecules.

### RELATED APPLICATIONS

This application claims priority under 35 U.S.C. §119 to U.S. Provisional Patent Application Serial No. 60/396,919, filed Jul. 17, 2002, which is hereby incorporated by reference.

### FIELD OF THE INVENTION

The invention provides new compositions and methods of use thereof for labeling and analyzing polymers such as nucleic acid molecules.

### BACKGROUND OF THE INVENTION

Many technologies relating to genomic sequencing and analysis require site-specific labeling of nucleic acid molecules. Most site-specific labeling is carried out using nucleic acid based probes that hybridize to their complementary sequences within a target molecule. The specificity of these probes will vary however depending upon their length, their sequence, the hybridization conditions, and the like. Moreover, because these probes are usually labeled with a detectable label such as a fluorophore or a radioactive label, they are expensive to synthesize. The ability to increase the specificity of these probes, and at the same time, use less of them would make labeling reactions more efficient and less expensive to run.

### SUMMARY OF THE INVENTION

The invention relates broadly to the use of particular nucleic acid containing conjugates for, inter alia, labeling and analyzing polymers, such as nucleic acids. These conjugates all commonly contain a polymer binding agent. In preferred embodiments, the polymer binding agent is a nucleic acid binding agent such as a nucleic acid binding enzyme. The invention is based, in part, on the discovery that a nucleic acid probe (referred to herein as “a nucleic acid tag molecule”) binds more efficiently to its target when it is used together with a nucleic acid binding agent. The nucleic acid binding agent, which preferably binds the nucleic acid molecule relatively non-specifically, concentrates the nucleic acid tag molecule in the vicinity of the target polymer to be labeled and/or analyzed. Therefore, less nucleic acid tag molecule is required to label or analyze the target polymer.

In one aspect, the invention provides a method for labeling a polymer. The method involves contacting the polymer with a conjugate comprising a nucleic acid tag molecule and a nucleic acid binding agent, allowing the nucleic acid binding agent to bind to the polymer, and allowing the nucleic acid tag molecule to bind specifically to the polymer. The method optionally contains the further step of determining a pattern of binding of the conjugate to the polymer.

The invention provides several aspects which share a number of identical embodiments. These embodiments are listed below and are intended (unless otherwise explicitly recited) to apply equally to all aspects provided herein.

Thus, in one embodiment, the nucleic acid binding agent is able to translocate along the length of the polymer. To translocate includes to move processively or non-processively along the length of a polymer. In some embodiments, the nucleic acid binding agent binds to the polymer non-specifically. In other embodiments, although the nucleic acid binding agent is normally capable of binding to the polymer in a specific (e.g., a sequence-specific manner), the conditions of binding are modified such that the binding of the agent to the polymer is relatively non-specific.

In important embodiments, the polymer is a nucleic acid molecule, and can be a non-in vitro amplified nucleic acid molecule. The polymer may be DNA or RNA, but it is not so limited.

The pattern of binding of the conjugate to the polymer may be determined using a variety of systems including a linear polymer analysis system. In some embodiments, the linear polymer analysis system is a single polymer analysis system. The nucleic acid molecule or the binding of the tag molecule to the nucleic acid molecule can be analyzed using a method selected from the group consisting of Gene Engine™, optical mapping, and DNA combing. The Gene Engine™ system is described in published PCT Patent Applications WO98/35012, WO00/09757 and WO01/13088, published on Aug. 13, 1998, Feb. 24, 2000 and Feb. 22, 2001 respectively, and in U.S. Pat. No. 6,355,420 B1 issued on Mar. 12, 2002, all of which are incorporated herein by reference in their entirety. Alternatively, the pattern may be determined using fluorescence in situ hybridization (FISH). Those of skill in the art will be aware of other systems that can be employed to determine the pattern of binding of the conjugate to the polymer.

In one embodiment, the nucleic acid tag molecule is selected from the group consisting of a peptide nucleic acid (PNA), a locked nucleic acid (LNA), a DNA, an RNA, a bisPNA, a pseudocomplementary PNA, and a LNA-DNA co-polymer, although it is not so limited. The nucleic acid tag molecule may be of any length, but in some preferred embodiments, it is 5-50 residues in length, and in even more preferred embodiments, it is 5-25 residues in length. The nucleic acid tag molecule is preferably a nucleic acid itself and therefore is composed of nucleotide units.

The nucleic acid tag molecule may be one that is capable of binding to the target polymer using Watson-Crick or Hoogsteen hybridization. The Watson-Crick bonds result in the formation of a double stranded complex as one strand of the nucleic acid target is displaced, while the Hoogsteen bonds result in the formation of a triple stranded complex since there is no need for displacement of the strands of the nucleic acid. In some important embodiments, a single nucleic acid tag molecule can bind to the target nucleic acid molecule by both Watson-Crick and Hoogsteen bonds, such as for example can occur if the tag molecule is a bisPNA. Various types of hybridization are described in Sinden R. R., DNA Structure and Function Academic Press, pp. 217-225 (1994). PNA and bisPNA hybridization is discussed in greater detail in Nielsen, P. E. et al., Peptide Nucleic Acids, Protocols and Applications, Norfolk: Horizon Scientific Press p. 1-19 (1999); and Kuhn, H. et al., J. Mol. Biol. 286:1337-1345 (1999).

The nucleic acid tag molecule and the nucleic acid binding agent are conjugated to each other either directly or indirectly. Indirect conjugation refers to the existence of a linker or spacer molecule in between the nucleic acid tag molecule and the nucleic acid binding agent. In preferred embodiments, the nucleic acid tag molecule and the nucleic acid binding agent are covalently conjugated to each other.

In important embodiments, the nucleic acid binding agent is an enzyme. The enzyme may be selected from the group consisting of a DNA polymerase, an RNA polymerase, a DNA repair enzyme, a helicase, a nuclease such as a restriction endonuclease, and a ligase, but it is not so limited. In important embodiments, the enzyme lacks the ability to modify the nucleic acid tag molecule or the polymer.

Depending upon the embodiment, the nucleic acid tag molecule and/or the nucleic acid binding agent and/or the polymer are labeled with a detectable moiety. The polymer is preferably labeled with a backbone specific label. In embodiments in which the nucleic acid tag molecule and the nucleic acid binding molecule are both labeled, their detectable moieties may be identical, or they may be different. Additionally, the detectable moieties may be detected using different detection systems. The nucleic acid binding agent may be detected indirectly, such as for example, using an antibody or an antibody fragment specific for the nucleic acid binding agent.

In some embodiments, the detectable moiety is selected from the group consisting of an electron spin resonance molecule (e.g., nitroxyl radicals), a fluorescent molecule, a chemiluminescent molecule, a radioisotope, an enzyme substrate, a biotin molecule, an avidin molecule, an electrical charge transferring molecule, a semiconductor nanocrystal, a semiconductor nanoparticle, a colloid gold nanocrystal, a ligand, a microbead, a magnetic bead, a paramagnetic particle, a quantum dot, a chromogenic substrate, an affinity molecule, a protein, a peptide, nucleic acid, a carbohydrate, an antigen, a hapten, an antibody, an antibody fragment, and a lipid.

In related embodiments, the detectable moiety is detected using a detection system. The detection system may be non-electrical in nature (such as a photographic film detection system), or it may be electrical in nature (such as a charge coupled device (CCD) detection system), but is not so limited. In some embodiments, the detection system is selected from the group consisting of a charge coupled device detection system, an electron spin resonance detection system, a fluorescent detection system, an electrical detection system, a photographic film detection system, a chemiluminescent detection system, an enzyme detection system, an atomic force microscopy (AFM) detection system, a scanning tunneling microscopy (STM) detection system, an optical detection system, a nuclear magnetic resonance (NMR) detection system, a near field detection system, and a total internal reflection (TIR) detection system.

In still other embodiments, the nucleic acid tag molecule is labeled with an agent such as a therapeutic agent. In one embodiment, the agent is able to modify a nucleic acid molecule and can include a methylase, a nuclease, and the like. The agent may also include inhibitors, activators, and regulators of DNA transcription. In one embodiment, the agent is one that cleaves a nucleic acid molecule. In some embodiments, the agent is a photocleaving agent.

In another aspect, the invention provides a system for optically analyzing a polymer. This system comprises an optical source for emitting optical radiation; an interaction station for receiving the optical radiation and for receiving a polymer that is exposed to the optical radiation to produce detectable signals; and a processor constructed and arranged to analyze the polymer based on the detected radiation including the signals. As described in the above aspect of the invention, the polymer is bound to a conjugate comprising a nucleic acid tag molecule and a nucleic acid binding agent.

In one embodiment, the interaction station includes a localized radiation spot. In a further embodiment, the system further comprises a microchannel that is constructed to receive and advance the polymer units through the localized radiation spot, and which optionally may produce the localized radiation spot. In another embodiment, the system further comprises a polarizer, wherein the optical source includes a laser constructed to emit a beam of radiation and the polarizer is arranged to polarize the beam. While laser beams are intrinsically polarized, certain diode lasers would benefit from the use of a polarizer. In some embodiments, the localized radiation spot is produced using a slit located in the interaction station. The slit may have a slit width in the range of 1 nm to 500 nm, or in the range of 10 nm to 100 nm. In some embodiments, the polarizer is arranged to polarize the beam prior to reaching the slit. In other embodiments, the polarizer is arranged to polarize the beam in parallel to the width of the slit.

In yet another embodiment, the optical source is a light source integrated on a chip. Excitation light may also be delivered using an external fiber or an integrated light guide. In the latter instance, the system would further comprise a secondary light source from an external laser that is delivered to the chip.

The polymer is bound, preferably specifically, to the conjugate of the nucleic acid tag molecule and the nucleic acid binding agent.

In still another aspect, the invention provides another method for analyzing a polymer. This method comprises generating optical radiation of a known wavelength to produce a localized radiation spot; passing a polymer through a microchannel; irradiating the polymer at the localized radiation spot; sequentially detecting radiation resulting from interaction of the polymer with the optical radiation at the localized radiation spot; and analyzing the polymer based on the detected radiation. The polymer is bound, preferably specifically, to a conjugate of a nucleic acid tag molecule and a nucleic acid binding agent. In one embodiment, the nucleic acid tag molecule of the conjugate binds specifically, to the polymer and the nucleic acid binding agent binds non-specifically to the polymer.

In one embodiment, the method further employs an electric field to pass the nucleic acid molecule through the microchannel. In another embodiment, detecting includes collecting the signals over time while the nucleic acid molecule is passing through the microchannel.

In yet another aspect, the invention provides a method for analyzing a nucleic acid molecule. This method comprises exposing a nucleic acid molecule to a conjugate of a nucleic acid tag molecule and a nucleic acid binding enzyme, allowing the nucleic acid binding enzyme to bind to the nucleic acid molecule, allowing the nucleic acid tag molecule to bind to the nucleic acid molecule in a sequence specific manner, and determining a pattern of binding of the conjugate to the nucleic acid molecule.

In one embodiment, the pattern of conjugate binding to the polymer is determined using a linear polymer analysis system (e.g., a direct linear analysis system). In a related embodiment, the linear polymer analysis system comprises exposing the polymer to a station to produce a signal arising from the binding of the conjugate to the polymer, and detecting the signal using a detection system incorporated into the linear polymer analysis system.

In another aspect, the invention provides a composition comprising a conjugate of a nucleic acid tag molecule and a nucleic acid binding enzyme, wherein a detectable moiety is present on the nucleic acid binding enzyme. In one embodiment, the nucleic acid tag molecule is labeled with a second detectable moiety. Preferably, the nucleic acid binding agent is not the detectable moiety.

In a similar aspect, the invention provides a composition comprising a conjugate of a nucleic acid tag molecule and a nucleic acid binding enzyme, wherein a detectable moiety is present on the nucleic acid tag molecule. In one embodiment, the nucleic acid binding enzyme is labeled with a second detectable moiety. In one embodiment, the nucleic acid binding enzyme is selected from the group consisting of a DNA polymerase, an RNA polymerase, a DNA repair enzyme, a helicase, a nuclease such as a restriction endonuclease, and a ligase.

In yet another aspect, the invention provides a method for analyzing a polymer comprising contacting the polymer with a conjugate comprising a nucleic acid tag molecule and a nucleic acid binding agent, allowing the nucleic acid binding agent to bind to the polymer, and allowing the nucleic acid tag molecule to bind specifically to the polymer. The nucleic acid binding agent is selected from the group consisting of a DNA repair enzyme, a helicase, a nuclease such as a restriction endonuclease, and a ligase.

In another aspect, the invention provides a method for analyzing a polymer comprising contacting the polymer with a conjugate comprising a nucleic acid tag molecule and a nucleic acid binding agent, allowing the nucleic acid binding agent to bind to and translocate along the polymer, and allowing the nucleic acid tag molecule to bind specifically to the polymer. In one embodiment, the nucleic acid binding agent binds to the polymer non-specifically. In another embodiment, the method further comprises determining a pattern of binding of the conjugate to the polymer.

These and other embodiments of the invention will be described in greater detail herein.

### BRIEF DESCRIPTION OF THE DRAWINGS

FIG. 1 is a schematic illustrating the conjugation of a nucleic acid binding agent (labeled “E”) and a nucleic acid tag molecule (labeled “PNA”), and subsequent scanning of a target nucleic acid molecule (labeled “DNA”).

FIG. 2 demonstrates examples of conjugation that are possible between fluorescent groups (R1 and R2) to protein surface amino (a), carboxylic (b), and thiol (c) groups with isothiocyanine, carbodiimide, and alkyl bromide, respectively.

FIG. 3 is a representation of the chemical structure of a peptide nucleic acid (PNA). The peptide bond formed during PNA synthesis is boxed.

FIG. 4 is a schematic showing looped structures formed on dsDNA following bisPNA invasion. Shown are the P loop (top panel), a merged or extended P loop (second panel), a PD loop with linear oligonucleotide (third panel), and an “earring” complex (bottom panel).

FIG. 5 shows the complex of dsDNA with a pair of pcPNAs hybridized thereto. Also shown are the structures of adenine, thymine, 2,6-diaminopurine, and ⁵U-2-thiouracil.

FIG. 6 is a representation of the chemical structure of a locked nucleic acid (LNA).

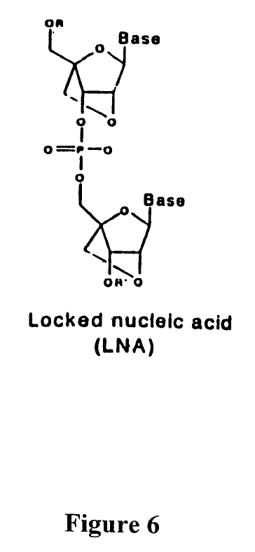

### DETAILED DESCRIPTION OF THE INVENTION

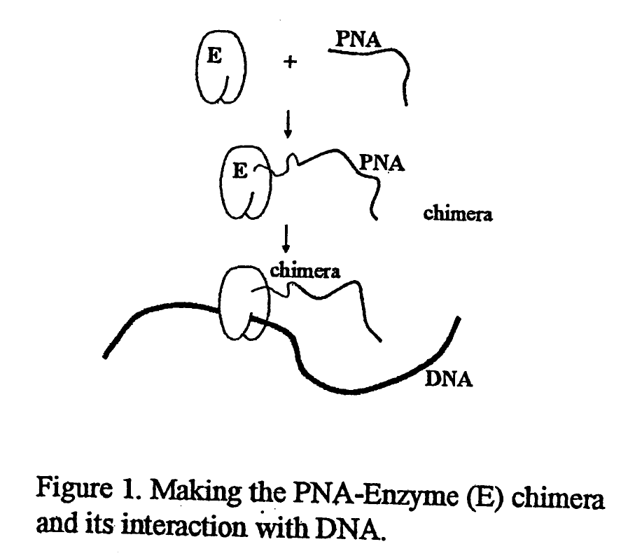

The invention is based, in part, on the discovery that the efficiency, stability and/or specificity of nucleic acid tag molecule binding to a target nucleic acid can be increased if the tag molecule is conjugated with a nucleic acid binding agent such as a nucleic acid binding enzyme. The conjugation of the tag molecules with the nucleic acid binding agent therefore overcomes some of the limitations encountered when using tag molecules alone to label and analyze nucleic acid molecules. Examples of these limitations include non-specific binding to reaction vessels, slow hybridization kinetics, aggregation of the target nucleic acid molecule induced by the tag molecule, difficulty and expense of labeling certain tag molecules, etc. The invention provides conjugate compositions as well as methods and systems for using the conjugates to label and analyze polymers such as nucleic acid molecules. These conjugates surprisingly overcome the afore-mentioned limitations. A schematic representation of the conjugate and its binding to a nucleic acid target are provided in FIG. 1.

The compositions and methods provided herein allow for a nucleic acid tag molecule (i.e., a sequence-specific probe) to be positioned close to a target nucleic acid molecule, thereby increasing its hybridization rate with the target nucleic acid. The methods also use less nucleic acid tag molecule since it is concentrated near the nucleic acid target, rather than free-slowing in the reaction solution.

The invention in one aspect intends to label and analyze target polymers that are nucleic acid molecules. It is not so limited, however, and could be used to label and analyze non-nucleic acid polymers. With the advent of aptamer technology, it is possible to use nucleic acid based probes (i.e., nucleic acid tag molecules) in order to recognize and bind a variety of compounds, including peptides and carbohydrates, in a structurally, and thus sequence, specific manner.

“Sequence specific” when used in the context of a nucleic acid molecule means that the tag molecule recognizes a particular linear arrangement of nucleotides or derivatives thereof. An analogous definition applies to non-nucleic acid polymers. In preferred embodiments, the linear arrangement includes contiguous nucleotides or derivatives thereof that each bind to a corresponding complementary nucleotide on the target nucleic acid. In some embodiments, however, the sequence may not be contiguous as there may be one, two, or more nucleotides that do not have corresponding complementary residues on the target.

The nucleic acid molecules used as targets may be DNA, or RNA, or amplification products or intermediates thereof, including complementary DNA (cDNA). The nucleic acid molecules can be directly harvested and isolated from a biological sample (such as a tissue or a cell culture) without the need for prior amplification using techniques such as polymerase chain reaction (PCR).

The sensitivity of methods provided herein allows single nucleic acid molecules to be analyzed individually. The nucleic acid molecules may be single stranded and double stranded nucleic acids. Harvest and isolation of nucleic acid molecules are routinely performed in the art and suitable methods can be found in standard molecular biology textbooks (e.g., such as Maniatis' Handbook of Molecular Biology). DNA includes genomic DNA (such as nuclear DNA and mitochondrial DNA), as well as in some instances cDNA. In important embodiments, the nucleic acid molecule is a genomic nucleic acid molecule. In related embodiments, the nucleic acid molecule is a fragment of a genomic nucleic acid molecule. The size of the nucleic acid molecule is not critical to the invention and it generally only limited by the detection system used.

In important embodiments of the invention, the nucleic acid molecule is a non in vitro amplified nucleic acid molecule. As used herein, a “non in vitro amplified nucleic acid molecule” refers to a nucleic acid molecule that has not been amplified in vitro using techniques such as polymerase chain reaction or recombinant DNA methods. A non in vitro amplified nucleic acid molecule may however be a nucleic acid molecule that is amplified in vivo (in the biological sample from which it was harvested) as a natural consequence of the development of the cells in vivo. This means that the non in vitro nucleic acid molecule may be one which is amplified in vivo as part of locus amplification, which is commonly observed in some cell types as a result of mutation or cancer development.

The size of the target nucleic acid molecule is not limiting. It can be several nucleotides in length, several hundred, several thousand, or several million nucleotides in length. In some embodiments, the nucleic acid molecule may be the length of a chromosome.

The term “nucleic acid” is used herein to mean multiple nucleotides (i.e. molecules comprising a sugar (e.g. ribose or deoxyribose) linked to an exchangeable organic base, which is either a substituted pyrimidine (e.g. cytosine (C), thymidine (T) or uracil (U)) or a substituted purine (e.g. adenine (A) or guanine (G)). “Nucleic acid” and “nucleic acid molecule” are used interchangeably. As used herein, the terms refer to oligoribonucleotides as well as oligodeoxyribonucleotides. The terms shall also include polynucleosides (i.e. a polynucleotide minus a phosphate) and any other organic base containing polymer. Nucleic acid molecules can be obtained from existing nucleic acid sources (e.g., genomic or cDNA), or by synthetic means (e.g. produced by nucleic acid synthesis).

The conjugates of the invention comprise a nucleic acid tag molecule. As used herein, a nucleic acid tag molecule is a molecule that is able to recognize and bind to a specific nucleotide sequence within a target nucleic acid molecule (i.e., the nucleic acid molecule intended to be labeled and/or analyzed).

Preferably, the nucleic acid tag molecules of the invention are not antisense nucleic acid molecules. As used herein, an antisense nucleic acid molecule is a nucleic acid that is an oligoribonucleotide, oligodeoxyribonucleotide, modified oligoribonucleotide, or modified oligodeoxyribonucleotide which hybridizes under physiological conditions to DNA comprising a particular gene or to an mRNA transcript of that gene and, thereby, inhibits the transcription of that gene and/or the translation of that mRNA. The antisense molecules are designed so as to interfere with transcription or translation of a target gene upon hybridization with the target gene or transcript.

The conjugates of the invention may be referred to herein as “chimeric tags” however they are not to be confused with the term nucleic acid tag molecule which refers solely to one component of the conjugates.

The nucleic acid tag molecules of the invention can themselves be nucleic acids or derivatives thereof. Such tag molecules can include substituted purines and pyrimidines such as C-5 propyne modified bases (Wagner et al., Nature Biotechnology 14:840-844, 1996). Purines and pyrimidines include but are not limited to adenine, cytosine, guanine, thymidine, 5-methylcytosine, 2-aminopurine, 2-amino-6-chloropurine, 2,6-diaminopurine, hypoxanthine, 2-thiouracil, pseudoisocytosine, and other naturally and non-naturally occurring nucleobases, substituted and unsubstituted aromatic moieties. Other such modifications are well known to those of skill in the art.

The tag molecules also encompass substitutions or modifications, such as in the bases and/or sugars. For example, they include nucleic acids having backbone sugars which are covalently attached to low molecular weight organic groups other than a hydroxyl group at the 3′ position and other than a phosphate group at the 5′ position. Thus, modified nucleic acids may include a 2′-O-alkylated ribose group. In addition, modified nucleic acids may include sugars such as arabinose instead of ribose. Thus the nucleic acids may be heterogeneous in backbone composition thereby containing any possible combination of polymer units linked together such as peptide nucleic acids (which have amino acid backbone with nucleic acid bases, and which are discussed in greater detail herein). In some embodiments, the nucleic acids are homogeneous in backbone composition.

When the conjugates of the invention are used in vivo e.g., added to live cells or tissues containing endo- and ex-nucleases, it may be preferable to use tag molecules that are resistant to degradation from such enzymes. A “stabilized nucleic acid tag molecule” shall mean a tag molecule that is relatively resistant to in vivo degradation (e.g. via an exo- or endonuclease).

It is to be understood that any nucleic acid analog that is capable of recognizing a nucleic acid molecule with structural or sequence specificity can be used as a nucleic acid tag molecule. In most instances, the nucleic acid tag molecules will form at least a Watson-Crick bond with the nucleic acid molecule. In other instances, the nucleic acid tag molecule can form a Hoogsteen bond with the nucleic acid molecule, thereby forming a triplex with the target nucleic acid. A nucleic acid sequence that binds by Hoogsteen binding enters the major groove of a nucleic acid target and hybridizes with the bases located there. Examples of these latter tag molecules include molecules that recognize and bind to the minor and major grooves of nucleic acids (e.g., some forms of antibiotics). In preferred embodiments, the nucleic acid tag molecules can form both Watson-Crick and Hoogsteen bonds with the target nucleic acid molecule. BisPNA tag molecules, discussed below, are capable of both Watson-Crick and Hoogsteen binding to a nucleic acid molecule. In most embodiments, tag molecules with strong sequence specificity are preferred.

In preferred embodiments, the nucleic acid tag molecule is a peptide nucleic acid (PNA), a bisPNA clamp, a pseudocomplementary PNA, a locked nucleic acid (LNA), DNA, RNA, or co-polymers of the above such as DNA-LNA co-polymers.

PNAs are DNA analogs having their phosphate backbone replaced with 2-aminoethyl glycine residues linked to nucleotide bases through glycine amino nitrogen and methylenecarbonyl linkers. PNAs can bind to both DNA and RNA targets by Watson-Crick base pairing, and in so doing form stronger hybrids that would be possible with DNA or RNA based tag molecules.

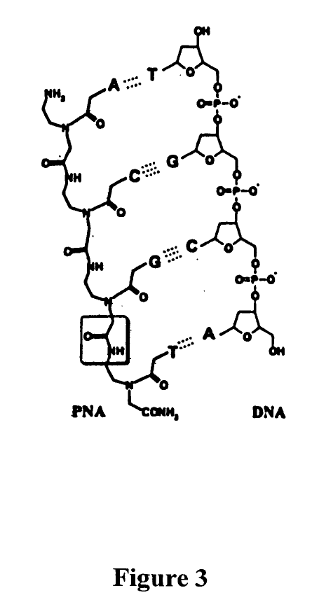

Peptide nucleic acid is synthesized from monomers connected by a peptide bond (Nielsen, P. E. et al. Peptide Nucleic Acids Protocols and Applications, Norfolk: Horizon Scientific Press, p. 1-19 (1999)), as shown in FIG. 3. It can be built with standard solid phase peptide synthesis technology.

PNA chemistry and synthesis allows for inclusion of amino acids and polypeptide sequences in the PNA design. For example, lysine residues can be used to introduce positive charges in the PNA backbone, as described below. All chemical approaches available for the modifications of amino acid side chains are directly applicable to PNAs.

PNA has a charge-neutral backbone, and this attribute leads to fast hybridization rates of PNA to DNA (Nielsen, P. E. et al. Peptide Nucleic Acids, Protocols and Applications, Norfolk: Horizon Scientific Press, p. 1-19 (1999)). The hybridization rate can be further increased by introducing positive charges in the PNA structure, such as in the PNA backbone or by addition of amino acids with positively charged side chains (e.g., lysines). PNA can form a stable hybrid with DNA molecule. The stability of such a hybrid is essentially independent of the ionic strength of its environment (Orum, H. et al., BioTechniques 19(3):472-480 (1995)), most probably due to the uncharged nature of PNAs. This provides PNAs with the versatility of being used in vivo or in vitro. However, the rate of hybridization of PNAs that include positive charges is dependent on ionic strength, and thus is lower in the presence of salt.

Several types of PNA designs exist, and these include single strand PNA (ssPNA), bisPNA, pseudocomplementary PNA (pcPNA).

The structure of PNA/DNA complex depends on the particular PNA and its sequence. Single stranded PNA (ssPNA) binds to ssDNA preferably in antiparallel orientation (i.e., with the N-terminus of the ssPNA aligned with the 3′ terminus of the ssDNA) and with a Watson-Crick pairing. PNA also can bind to DNA with a Hoogsteen base pairing, and thereby forms triplexes with dsDNA (Wittung, P. et al., Biochemistry 36:7973 (1997)).

The presence of mismatches destabilizes PNA/DNA hybrids to a greater extent than DNA/DNA hybrids (Egholm, M. et al., Nature 365:566-568 (1993)). This increase in specificity can be compounded with the use of shorter PNA tag molecules.

Single strand PNA is the simplest of the PNA molecules. This PNA form interacts with nucleic acids to form a hybrid duplex via Watson-Crick base pairing. The duplex has different spatial structure and higher stability than dsDNA (Nielsen, P. E. et al. Peptide Nucleic Acids, Protocols and Applications, Norfolk: Horizon Scientific Press, p. 1-19 (1999)). However, when different concentration ratios are used and/or in presence of complimentary DNA strand, PNA/DNA/PNA or PNA/DNA/DNA triplexes can also be formed (Wittung, P. et al., Biochemistry 36:7973 (1997)). The formation of duplexes or triplexes additionally depends upon the sequence of the PNA. Thymine-rich homopyrimidine ssPNA forms PNA/DNA/PNA triplexes with dsDNA targets where one PNA strand is involved in Watson-Crick antiparallel pairing and the other is involved in parallel Hoogsteen pairing. Cytosine-rich homopyrimidine ssPNA preferably binds through Hoogsteen pairing to dsDNA forming a PNA/DNA/DNA triplex. If the ssPNA sequence is mixed, it invades the dsDNA target, displaces the DNA strand, and forms a Watson-Crick duplex. Polypurine ssPNA also forms triplex PNA/DNA/PNA with reversed Hoogsteen pairing.

BisPNA includes two strands connected with a flexible linker. One strand is designed to hybridize with DNA by a classic Watson-Crick pairing, and the second is designed to hybridize with a Hoogsteen pairing. The target sequence can be short (e.g., 8 bp), but the bisPNA/DNA complex is still stable as it forms a hybrid with twice as many (e.g., a 16 bp) base pairings overall. The bisPNA structure further increases specificity of their binding. As an example, binding to an 8 bp site with a tag having a single base mismatch results in a total of 14 bp rather than 16 bp.

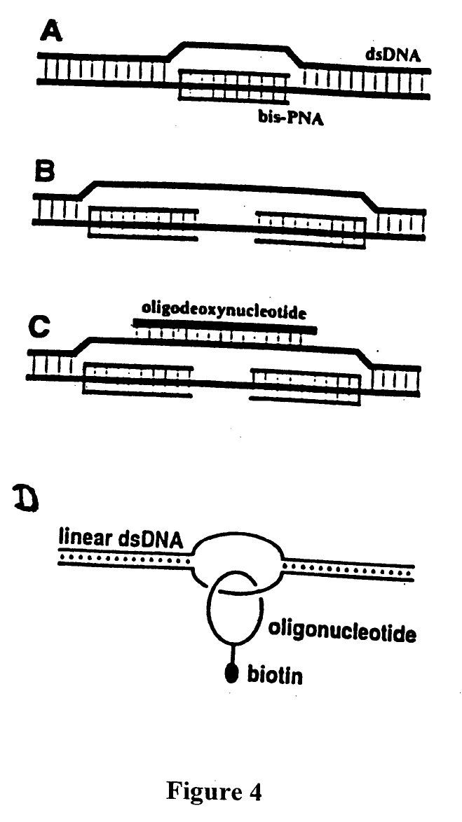

The current model assumes that on the first stage of hybridization the bisPNA molecule has its Hoogsteen strand bound to the target site, followed by the invasion of the Watson-Crick strand to form a triplex with one of the original DNA strands displaced (FIG. 4). To facilitate the second stage, the hybridization reaction is performed at elevated temperature to increase the frequency of DNA helix opening (i.e., localized melting). That mechanism increases the overall hybridization rate dramatically, since at the moment of DNA opening, the Watson-Crick strand of bisPNA is positioned to invade the helix.

Preferably, bisPNAs have homopyrimidine sequences, and even more preferably, cytosines are protonated to form a Hoogsteen pair to a guanosine. Therefore, bisPNA with thymines and cytosines is capable of hybridization to DNA only at pH below 6.5. The first restriction—homopyrimidine sequence only—is inherent to the mode of bisPNA binding. Pseudoisocytosine (J) can be used in the Hoogsteen strand instead of cytosine to allow its hybridization through a broad pH range (Kuhn, H., J. Mol. Biol. 286:1337-1345 1999)).

BisPNAs have multiple modes of binding to nucleic acids (Hansen, G. I. et al., J. Mol. Biol. 307(1):67-74 (2001)). One isomer includes two bisPNA molecules instead of one. It is formed at higher bisPNA concentration and has tendency to rearrange into the complex with a single bisPNA molecule. Other isomers differ in positioning of the linker around the target DNA strands. All the identified isomers still bind to the same binding site/target.

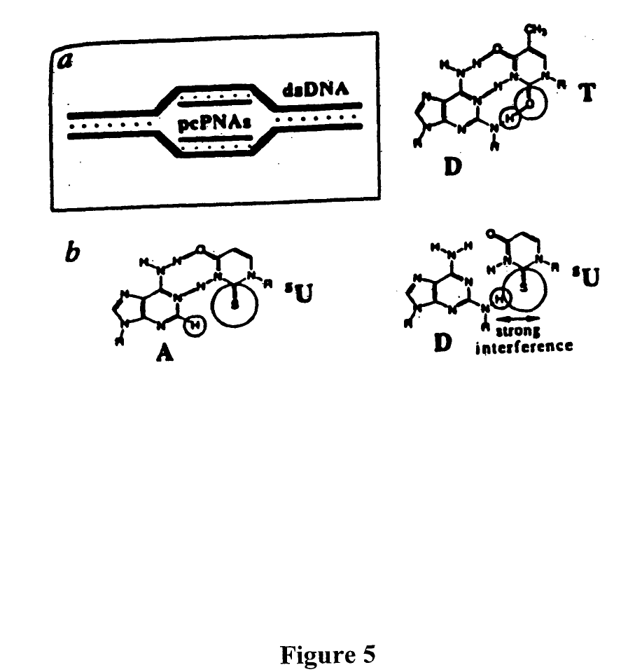

Pseudocomplementary PNA (pcPNA) (Izvolsky, K. I. et al., Biochemistry 10908-10913 (2000)) involves two single stranded PNAs added to dsDNA. One pcPNA strand is complementary to the target sequence, while the other is complementary to the displaced DNA strand (FIG. 5). As the PNA/DNA duplex is more stable, the displaced DNA generally does not restore the dsDNA structure. The PNA/PNA duplex is more stable than the DNA/PNA duplex and the PNA components are self-complementary because they are designed against complementary DNA sequences. Hence, the added PNAs would rather hybridize to each other. To prevent the self-hybridization of pcPNA units, modified bases are used for their synthesis including 2,6-diamiopurine (D) instead of adenine and 2-thiouracil (^{S}U) instead of thymine. While D and ^{S}U are still capable of hybridization with T and A respectively, their self-hybridization is sterically prohibited (FIG. 5).

This PNA construct also delivers two base pairs per every nucleotide of the target sequence. Hence, it can bind to short sequences similar to those that are bisPNA targets. The pcPNA strands are not connected by a hinge, and they have different sequences.

Hybridization of pcPNA can be less efficient than that of bisPNA because it needs three molecules to form the complex. However, the pseudocomplementary stands can be connected by a sufficiently long and flexible hinge.

Another bisPNA-based approach involves use of the displaced DNA strand (Demidov, V. V. et al., Methods: A Companion to Methods in Enzymology 23(2):123-131 (2001)). If the second bisPNA is hybridized close enough to the first one, then a run of DNA (up to 25 bp) is displaced, forming an extended P-loop (FIG. 4). This run is long enough to be tagged. This combination is referred to as a PD-loop (Demidov, V. V. et al., Methods: A Companion to Methods in Enzymology 23(2):123-131 (2001)). Other applications for the opening are also designed including topological labels or “earrings” (FIG. 4). Tagging based on PD-loop has important advantages, including increased specificity.

In some embodiments, conjugates comprising tag molecules that are PNA are preferred because it has been reported that PNA/DNA hybrids are more stable that DNA/DNA hybrids. This is important, particularly when analyzing double stranded nucleic acids such as genomic DNA (especially if performed in situ) because the PNA tag molecule will not be displaced by the complementary DNA strand of the target molecule. Accordingly, the PNA/DNA complex can exist for days at room temperature. Moreover, PNA-based tag molecules offer the advantages of efficient and specific hybridization, formation of stable complexes, flexible chemistry, and resistance against degradation by other enzymes.

In some embodiments, positive charges are incorporated into a tag molecule (such as a PNA tag molecule) in order to improve the interaction of such tag molecules with a DNA target. Such modification increases the hybridization rate due to electrostatic attraction of the positively charged tag molecule and the negatively charged backbone of the target nucleic acid molecule.

Locked nucleic acid (LNA) molecules form hybrids with DNA, which are at least as stable as PNA/DNA hybrids (Braasch, D. A. et al., Chem & Biol. 8(1):1-7(2001)). Therefore, LNA can be used just as PNA molecules would be. LNA binding efficiency can be increased in some embodiments by adding positive charges to it, as described herein. LNAs have been reported to have increased binding affinity inherently. When used in the conjugates of the invention, these LNAs can be concentrated in the region of the target nucleic acid molecule, thereby enhancing their binding to the target.

Commercial nucleic acid synthesizers and standard phosphoramidite chemistry are used to make LNA oligomers. Therefore, production of mixed LNA/DNA sequences is as simple as that of mixed PNA/peptide sequences. The stabilization effect of LNA monomers is not an additive effect. The monomer influences conformation of sugar rings of neighboring deoxynucleotides shifting them to more stable configurations (Nielsen, P. E. et al. Peptide Nucleic Acids, Protocols and Applications, Norfolk: Horizon Scientific Press, p. 1-19 (1999)). Also, lesser number of LNA residues in the sequence dramatically improves accuracy of the synthesis. Naturally, most of biochemical approaches for nucleic acid conjugations are applicable to LNA/DNA constructs.

The tag molecules can also be stabilized in part by the use of other backbone modifications. The invention intends to embrace in addition to the peptide and locked nucleic acids discussed herein, the use of the other backbone modifications such as but not limited to phosphorothioate linkages phosphodiester modified nucleic acids, combinations of phosphodiester and phosphorothioate nucleic acid, methylphosphonate, alkylphosphonates, phosphate esters, alkylphosphonothioates, phosphoramidates, carbamates, carbonates, phosphate triesters, acetamidates, carboxymethyl esters, methylphosphorothioate, phosphorodithioate, p-ethoxy, and combinations thereof.

Other backbone modifications, particularly those relating to PNAs, include peptide and amino acid variations and modifications. Thus, the backbone constituents of PNAs may be peptide linkages, or alternatively, they may be non-peptide linkages. Examples include acetyl caps, amino spacers such as O-linkers, amino acids such as lysine (particularly useful if positive charges are desired in the PNA), and the like. Various PNA modifications are known and tags incorporating such modifications are commercially available from sources such as Boston Probes, Inc.

One limitation of the stability of nucleic acid hybrids is the length of the tag molecule, with longer tag molecules leading to greater stability than shorter tag molecules. Notwithstanding this proviso, the tag molecules of the invention can be any length ranging from at least 4 nucleotides long to in excess of 1000 nucleotides long. In preferred embodiments, the tag molecules are 6-100 nucleotides in length, more preferably between 525 nucleotides in length, and even more preferably 5-12 nucleotides in length. The length of the tag molecule can be any length of nucleotides between and including the ranges listed herein, as if each and every length was explicitly recited herein. It should be understood that not all residues of the tag molecule need hybridize to complementary residues in the target nucleic acid molecule. For example, the tag molecule may be 50 residues in length, yet only 25 of those residues hybridize to the target nucleic acid. Preferably, the residues that hybridize are contiguous with each other.

The tag molecules are preferably single stranded, but they are not so limited. For example, when the tag molecule is a bisPNA it can adopt a secondary structure with the nucleic acid target resulting in a triple helix conformation, with one region of the bisPNA clamp forming Hoogsteen bonds with the backbone of the target molecule and another region of the bisPNA clamp forming Watson-Crick bonds with the nucleotide bases of the target molecule.

Tag molecules that are bisPNA clamps can bind to target nucleic acid molecules in the absence of displacement of one DNA strand since these clamps hybridize directly to double stranded DNA without melting or opening of the double stranded helix.

The length of the tag molecule (and the target sequence) determines the specificity of binding. The energetic cost of a single mismatch between the tag molecule and the nucleic acid target is relatively higher for shorter sequences than for longer ones. Therefore, hybridization of small sequences is more specific than is hybridization of longer sequences because the longer sequences can embrace mismatches and still continue to bind to the target depending on the conditions. One potential limitation to the use of shorter tag molecules however is their inherently lower stability at a given temperature and salt concentration. In order to avoid this latter limitation, bisPNA tag molecules can be used which allow both shortening of the target sequence and sufficient hybrid stability in order to detect tag molecule (and thus conjugate) binding to the nucleic acid molecule being analyzed. BisPNAs can be longer than standard nucleic acid tags although capable of binding to shorter target sequences.

Another consideration in determining the appropriate tag molecule length is whether the sequence to be detected is unique or not. If the method is intended only to sequence the target nucleic acid, then unique sequences may not be that important provided they are sufficiently spaced apart from each other to be able to detect the signal from each binding event separately from the others. That is, the sequence should randomly occur at distances that can be discerned as separate sites along the polymer, otherwise, the signals merge. As long as the location of binding of separate conjugates along the length of a target polymer can be distinguished, it should be clear that a greater resolution is possible using smaller tag molecules.

In one embodiment, a library of tag molecules (and corresponding conjugates) is generated of an identical length. The library will preferably contain every possible combination of sequence for that particular length. It should also be clear that such libraries will be smaller for shorter tag sequences than for longer tag sequences because there are fewer combinations possible.

If on the other hand, the method is used to test for the presence of a mutant sequence such as a translocation event, or a genetic mutation associated with a particular disorder or predisposition to a disorder, then the tag molecule may be longer in order to capture only its true complement.

The methods of the invention embrace the use of one or more conjugates. In preferred embodiments, the conjugates differ on in terms of the tag molecule they carry. That is, the tag molecule is different, and thus binds to a different sequence along the length of the target nucleic acid. Also preferably, different conjugates are labeled differently so that it is possible to distinguish the binding of each from the other. In this way, it is possible to derive a greater amount of sequence information.

Preferably, the nucleic acid tag molecules recognize and bind to sequences within the target polymer (i.e., the polymer being labeled and/or analyzed). If the polymer is itself a nucleic acid molecule, then the nucleic acid tag molecule preferably recognizes and binds by hybridization to a complementary sequence within the target nucleic acid. The specificity of binding can be manipulated based on the hybridization conditions. For example, salt concentration and temperature can be modulated in order to vary the range of sequences recognized by the nucleic acid tag molecules.

In some embodiments, the nucleic acids to be analyzed are from non-microbial sources, and thus the tag molecules are specific for non-microbial nucleotide sequences. As used herein, a non-microbial nucleotide sequence is a sequence that is found only in microbial species and not in non-microbial species. As used herein, a microbial species is a bacteria, a virus, a fungus, or a parasite. In other embodiments, the tag molecules are specific for sequences found only in bacteria, viruses (e.g., HIV), fungi or parasites.

In some embodiments, the invention embraces the use of tag molecules that recognize and bind to the minor and/or major grooves of the nucleic acid molecule. Still this recognition is dependent upon the ultimate sequence of the nucleic acid molecule, and thus binding of the tag molecule imparts information regarding the sequence of the nucleic acid. An example of a class of compounds that binds to nucleic acid grooves is antibiotics.

In some instances, the nucleic acid tag molecules of the invention can be synthesized to have groups other than nucleotides attached thereto. For example, the tag molecules can also comprise one or more reactive groups (e.g., for conjugation to the nucleic acid binding agent or to a linker, as described below), one or more amino acids (e.g., for reaction with linkers), or detectable moieties (as described below).

The conjugates of the invention further comprise a nucleic acid binding agent. As used herein, a nucleic acid binding agent is an agent that binds to a nucleic acid molecule and is able to move along the length of the nucleic acid molecule, but is relatively insensitive to the sequence of the nucleic acid. In this way, the nucleic acid binding agent is able to scan the length of the nucleic acid molecule allowing the tag molecule to contact its complement on the nucleic acid molecule. It is preferred that the ultimate location of the conjugate on the nucleic acid molecule is a function of the specificity of the tag molecule rather than the binding agent.

Preferably, the nucleic acid binding agent is a nucleic acid binding enzyme. It may be but is not limited to a DNA polymerase including Klenow fragment and reverse transcriptase, an RNA polymerase, a DNA repair enzyme, DNase 1, a helicase, nucleases such as restriction endonuclease (preferably engineered to remove nuclease activity but retain scanning ability), a topoisomerase, a ligase, a methylase such as DNA methyltransferase (in some embodiments, engineered to remove methylase activity, but retain scanning ability), DNA repair enzymes and machinery, and the like. An example of a nucleic acid binding agent that binds to single stranded nucleic acids is SPP1-encoded replicative DNA helicase gene 40 product (G40P).

Although not intending to be bound by any particular mechanism, it is believed that in one aspect the invention exploits the ability of the nucleic acid binding agent to bind a nucleic acid molecule in a relatively sequence non-specific manner, and to translocate along the length of the nucleic acid molecule until the complement of the tag molecule is found. As used herein, a sequence non-specific manner refers to binding that is sequence independent. As used herein, the term “translocate” means that the nucleic acid binding agent moves along the length of a nucleic acid molecule. The binding agent can move along the nucleic acid molecule in a one-dimensional diffusion manner, or alternatively it can dissociate and reassociate with another region of the nucleic acid molecule. Translocate embraces both processive movement along the length of the nucleic acid molecule as well as non-processive movement along the length of the nucleic acid molecule. Processive movement means that the nucleic acid binding agent progressively moves along the length of a polymer without dissociating from it, while non-processive movement means that the nucleic acid binding agent randomly associates and dissociates with the polymer. Lifetimes of specifically and non-specifically bound enzymes have been reported to be about 0.1-10 seconds and 1 hour, respectively. (Taylor, J. R. et al., Anal. Chem. 72(9):1979-1986 (2000)).

It is also possible that the nucleic acid binding agent can destabilize and even distort a double stranded nucleic acid molecule (such as a double stranded DNA molecule). This has been reported for EcoRV by Sam, M. D. et al., Biochem. 38(20):6576-6586 (1999). This effect may further enhance hybridization of the tag molecule with the target nucleic acid molecule, with the result that the hybridization can be performed at even lower tag molecule concentration and/or at a decreased temperature. Both of these latter changes in turn can effectively decrease tag molecule (especially PNA) induced aggregation of the target nucleic acid molecule.

By conjugating the tag molecules of the invention to a nucleic acid binding agent such as a nucleic acid binding enzyme, it is possible to increase the stability and half-life of the above-noted hybrids. For example, shorter bisPNA tag molecules can be used since binding stability can be imparted by the nucleic acid binding agent. Moreover, the use of a nucleic acid binding agent effectively insures that all tag molecules will be concentrated in the vicinity of the nucleic acid molecule. This reduces the amount of tag molecule that must be used in order to label and analyze the polymer since little if any tag molecule is wasted.

Conjugation of the tag molecule to the nucleic acid binding agent also serves to increase the hybridization rate and time of hybridization between the tag molecule and the target polymer. The nucleic acid binding agent is intended to function as an anchor for the nucleic acid tag molecule, maintaining the tag molecule in the vicinity of the target nucleic acid molecule until it is able to find and bind to its complementary sequence. Sliding of the conjugate along the nucleic acid backbone facilitates interaction of the tag molecule with complementary target sites that would otherwise be hidden inside the nucleic acid secondary or tertiary structure. Such sites would generally be inaccessible to free tag molecules in solution.

In some embodiments, the enzyme is engineered such that it lacks the ability to modify the nucleic acid molecules being analyzed or the tag molecules of the conjugate.

While all of the foregoing enzymes have some level of specificity for particular sequences or structures of nucleic acid molecules, such specificity can be minimized in a number of ways, including the conditions at which binding and translocation are performed. Moreover, the invention also embraces that use of mutants of such enzymes that lack sequence specificity, although they are still capable of recognizing and binding to nucleic acids in general. For example, some nucleic acid binding enzymes have separate domains responsible for their binding to particular regions of nucleic acid molecule, and these domains can be mutated so that the enzyme binds non-specifically to a nucleic acid molecule. As yet another alternative, enzymes with some binding specificity can be used in such excess that all of their target sites are saturated, forcing the excess enzymes to bind at other sites.

In some preferred embodiments, the nucleic acid binding enzyme is capable non-specifically binding and translocating (e.g., “scanning”) along the length of a nucleic acid target. Agents that bind to specific sequences and/or structures (e.g., minor or major groove binding agents) are less desirable as nucleic acid binding agents than are agents that can translocate along the length of a nucleic acid molecule.

In embodiments in which the nucleic acid binding agent is an enzyme having nuclease activity, it is preferable that such nuclease activity be suppressed. This can be accomplished either chemically or by protein engineering. For example, restriction activity of restriction endonucleases can be suppressed by removal of divalent cations from hybridization solutions, since such enzymes are dependent upon divalent cations for their nuclease activity. The activity can also be suppressed by genetically engineering the protein to remove or reduce this activity. Such engineering can be directed, or random depending upon the level of knowledge of the protein structure and its nucleic acid sequence. If done randomly, the resultant clones should be screened for their ability to bind nucleic acids without cleavage. Such screens are routine to those of skill in the art.

In embodiments in which the nucleic acid binding enzyme is a polymerase, it may be desirable to remove not only the nuclease activity of such an enzyme but also its polymerase activity, so that it cannot synthesize new nucleic acid molecules. Preferably, the polymerase is not itself a detectable label in that its position is not detected through its ability to synthesize a nucleic acid molecule.

The nucleic acid binding agents of the invention can bind and scan along DNA or RNA molecules, or both. In some embodiments, the binding constants of such nucleic acid binding agents are in the range of 10⁹ M⁻¹ to 10¹³ M⁻¹. Because of this binding affinity, the nucleic acid binding agent will accumulate in the vicinity of a nucleic acid molecule, as will the tag molecule to which it is conjugated.

The nucleic acid binding enzymes can themselves be chimeric in nature i.e., composed or engineered from two or more different enzymes or proteins.

In preferred embodiments, the nucleic acid binding agent is not inherently a label. For example, the agent is not an enzyme that can be detected based on its catalytic activity. Rather, to be visualized and/or detected, the nucleic acid binding agent must have attached to it a detectable label or moiety. Thus, for example, if the nucleic acid binding agent is a polymerase such as a DNA polymerase, it has attached thereto a detectable moiety.

The conjugates are formed by linking the tag molecules to the nucleic acid binding agents (e.g., enzymes). This linkage can be covalent or non-covalent in nature, although covalent linkage is preferred. As used herein a conjugate is any physical linkage between the nucleic acid tag molecule and the nucleic acid binding agent. The conjugation of these two components should not however interfere with either the ability of the nucleic acid tag molecule to recognize and bind to its complementary sequence, or the ability of the nucleic acid binding agent to recognize and translocate along a nucleic acid molecule.

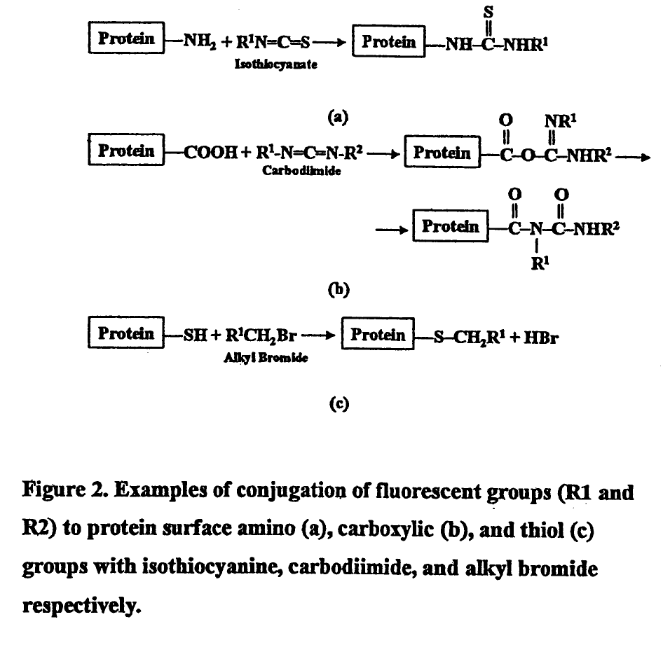

The most simple way to conjugate a nucleic acid tag molecule to a nucleic acid binding agent that is a protein is to use the surface groups of the binding agent. Sample chemical conjugation reactions are presented in FIG. 2. These groups (e.g., amino, carboxylic, and thiol) are usually part of amino acid side chains and usually are exposed to solvent. Other chemical approaches are available as well, and these are known to those of ordinary skill in the art.

To prevent cross-linking of nucleic acid, it is desirable to conjugate one tag molecule per binding agent. This can be achieved by attaching the tag molecule to the binding enzyme using a thiol group rather than an amino or a carboxylic group, both of which are very common in proteins. Moreover, attachment to an amino group may interfere with the ability of the nucleic acid binding enzyme to bind to the nucleic acid molecule because these groups are sometimes involved in nucleic acid binding. As an example, the active form of EcoRI has two subunits of molecular weight approximately 29 kD that include 20 lysine and 1-2 cysteine residues. (Modrich, P. et al., J. Biol. Chem. 251:5866-5874 (1976)). Lysines and cysteines have amino and thiol groups in their side chains respectively. If the EcoRI subunits are used, it may be preferable to attach the tag molecules to the cysteine residues since they are fewer in number, thus ensuring that only one tag molecule is attached to a given subunit.

Sorting of conjugates after conjugation is also possible. For example, conjugates in which the nucleic acid binding agent has been conjugated to a tag molecule via active amino groups, can be separated from conjugates in which the tags are conjugated via non-active amino groups. This separation can be carried out using, for example, affinity chromatography on a column with dsDNA fragments as the former conjugates which are incapable of binding to DNA will pass through the column unretarded, while the latter conjugates which can bind to DNA will be delayed and eluted in later fractions. Similarly, conjugates that comprise more than one tag molecule can be separated from those having only one tag molecule, for example, using HPLC.

It is also possible to manipulate the number and positions of thiol groups in enzymes by protein engineering without affecting the nucleic acid binding capacity of the enzyme.

Moreover, the linkage can include a linker molecule in between the tag molecule and the nucleic acid binding agent. It may be desirable, in some instances, to tether the tag molecule to the nucleic acid binding agent via a spacer or linker molecule. This can remove, for example, any problems that might arise from steric hindrance, wherein access by the tag molecule to it complementary sequence in the nucleic acid molecule is hindered. Preferably, the linker is sufficiently long and flexible to allow the tag molecule to interact with the target nucleic acid molecule.

These spacers can be any of a variety of molecules, preferably nonactive, such as straight or even branched carbon chains of C₁-C₃₀, saturated or unsaturated, phospholipids, amino acids, and in particular glycine, and the like, naturally occurring or synthetic. Additional spacers include alkyl and alkenyl carbonates, carbamates, and carbamides. These are all related and may add polar functionality to the spacers such as the C₁-C₃₀ previously mentioned.

A wide variety of spacers can be used, many of which are commercially available, for example, from sources such as Boston Probes, Inc. (now Applied Biosystems, Inc.). Spacers are not limited to organic spacers, and rather can be inorganic also (e.g., —O—Si—O—, or O—P—O). Additionally, they can be heterogeneous in nature (e.g., composed of organic and inorganic elements). Essentially, any molecule with reactive groups on its termini can be used as a spacer. Example of spacers include the linkers supplied by Boston Probes, Inc. including the E linker (which also functions as a solubility enhancer), the X linker which is similar to the E linker, the O linker which is a glycol linker, and the P linker which includes a primary aromatic amino group. Other suitable linkers are acetyl linkers, 4-aminobenzoic acid containing linkers, Fmoc linkers, 4-aminobenzoic acid linkers, 8-amino-3, 6-dioxactanoic acid linkers, succinimidyl maleimidyl methyl cyclohexane carboxylate linkers, succinyl linkers, and the like. Another example of a suitable linker is that described by Haralambidis et al. in U.S. Pat. No. 5,525,465, issued on Jun. 11, 1996.

The length of the spacer can vary depending upon the application and the nature of the nucleic acid binding agent and the tag molecule. In some important embodiments, it has a length of not greater than 100 nm, and in some preferred embodiments, it has a length of 1-10 nm.

The conjugations or modifications described herein employ routine chemistry, which is known to those skilled in the art of chemistry. The use of protecting groups and known linkers such as mono- and hetero-bifunctional linkers are documented in the literature (e.g., Hermanson, 1996) and will not be repeated here.

Specific examples of covalent bonds include those wherein bifunctional cross-linker molecules are used. The cross-linker molecules may be homo-bifunctional or heterobifunctional, depending upon the nature of the molecules to be conjugated. Homobifunctional cross-linkers have two identical reactive groups. Hetero-bifunctional cross-linkers are defined as having two different reactive groups that allow for sequential conjugation reaction. Various types of commercially available cross-linkers are reactive with one or more of the following groups: primary amines, secondary amines, sulphydryls, carboxyls, carbonyls and carbohydrates. Examples of amine-specific cross-linkers are bis(sulfosuccinimidyl) suberate, bis[2-(succinimidooxycarbonyloxy)ethyl] sulfone, disuccinimidyl suberate, disuccinimidyl tartarate, dimethyl adipimate-2 HCl, dimethyl pimelimidate-2 HCl, dimethyl suberimidate-2 HCl, and ethylene glycolbis-[succinimidyl-[succinate]]. Cross-linkers reactive with sulfhydryl groups include bismaleim idohexane, 1,4-di-[3′-(2′-pyridydithio)-propionamido)]butane, 1-[p-azidosalicylamido]-4-[iodoacetamido]butane, and N-[4-(p-azidosalicylamido)butyl]-3′-[2′-pyridyidithio]propionamide. Cross-linkers preferentially reactive with carbohydrates include azidobenzoyl hydrazine. Cross-linkers preferentially reactive with carboxyl groups include 4-[p-azidosalicylamido]butylamine. Heterobifunctional cross-linkers that react with amines and sulfhydryls include N-succinimidyl-3-[2-pyridyidithio]propionate, succinimidyl [4-iodoacetyl]aminobenzoate, succinimidyl 4-[N-maleimidomethyl] cyclohexane-1-carboxylate, m-maleimidobenzoyl-N-hydroxysuccinimide ester, sulfosuccinimidyl 6-[3-[2-pyridyidithio]propionamido]hexanoate, and sulfosuccinimidyl 4-[N-maleimidomethyl]cyclohexane-1-carboxylate. Heterobifunctional cross-linkers that react with carboxyl and amine groups include 1-ethyl-3-[[3-dimethylaminopropyl]carbodiimide hydrochloride. Heterobifunctional cross-linkers that react with carbohydrates and sulfhydryls include 4-[N-maleimidomethyl]-cyclohexane-1-carboxylhydrazide-2 HCl, 4-(4-N-maleimidophenyl)-butyric acid hydrazide-2 HCl, and 3-[2-pyridyidithio]propionyl hydrazide. The cross-linkers are bis-[β-4-azidosalicylamido)ethyl]disulfide and glutaraldehyde.

Amine or thiol groups may be added at any nucleotide of a synthetic nucleic acid so as to provide a point of attachment for a bifunctional cross-linker molecule. The nucleic acid may be synthesized incorporating conjugation-competent reagents such as Uni-Link AminoModifier, 3′-DMT-C6-Amine-ON CPG, AminoModifier II, N-TFA-C6-AminoModifier, C6-ThiolModifier, C6-Disulfide Phosphoramidite and C6-Disulfide CPG (Clontech, Palo Alto, Calif.).

In some embodiments, it may be desirable to attach the tag molecule to the nucleic acid binding agent by a bond that can be cleaved under certain conditions. For example, the bond can be one that cleaves under normal physiological conditions or that can be caused to cleave specifically upon application of a stimulus such as light, whereby the agent can be released, leaving only the tag molecule bound to the nucleic acid molecule being labeled or analyzed. Readily cleavable bonds include readily hydrolyzable bonds, for example, ester bonds, amide bonds and Schiff's base-type bonds. Bonds which are cleavable by light are known in the art. Using such linkages, it is possible to remove the nucleic acid binding agent from the conjugate following sequence specific binding to the nucleic acid molecule. In these latter embodiments, it is desirable that the nucleic acid tag molecule is labeled with a detectable moiety.

Noncovalent methods of conjugation may also be used. Noncovalent conjugation includes hydrophobic interactions, ionic interactions, Van der Waals (or dispersion) interactions, hydrogen bonding, etc. High affinity interactions such as biotin-avidin and biotin-streptavidin complexation, and antigen/hapten-immunoglobulin interactions, and receptor-ligand interactions are also envisioned. In one embodiment, a molecule such as avidin is attached to the nucleic acid binding agent, and its binding partner biotin is attached to the nucleic acid tag molecule.

The conjugates of the invention are labeled with detectable moieties. The moiety can be detected directly by its ability to emit and/or absorb light of a particular wavelength. A moiety can be detected indirectly by its ability to bind, recruit and, in some cases, cleave another moiety which itself may emit or absorb light of a particular wavelength. An example of indirect detection is the use of a first enzyme label which cleaves a substrate into visible products. The label may be of a chemical, peptide or nucleic acid nature although it is not so limited. Detectable moieties can be conjugated to conjugate using thiol, amino or carboxylic groups. Because it may be desirable to attach as many detectable labels to the conjugate or to either component of the conjugate as possible, such labels may be attached to amino or carboxylic groups which are common on proteins.

In preferred embodiments, the conjugates themselves are not detectable moieties (i.e., their presence cannot be detected because of an inherent feature of either component of the conjugate). As an example, the nucleic acid binding agent is preferably not itself a detectable moiety, meaning that it does not have an inherent enzymatic activity that can be used to detect its presence.

The detectable moieties described herein are referred to according to the systems by which they are detected. As an example, a flourophore molecule is a molecule that can be detected using a system of detection that relies on fluorescence.

Generally, the detectable moiety can be selected from the group consisting of an electron spin resonance molecule (such as for example nitroxyl radicals), a fluorescent molecule, a chemiluminescent molecule, a radioisotope, an enzyme substrate, a biotin molecule, a streptavidin molecule, a peptide, an electrical charge transferring molecule, a semiconductor nanocrystal, a semiconductor nanoparticle, a colloid gold nanocrystal, a ligand, a microbead, a magnetic bead, a paramagnetic particle, a quantum dot, a chromogenic substrate, an affinity molecule, a protein, a peptide, nucleic acid, a carbohydrate, an antigen, a hapten, an antibody, an antibody fragment, and a lipid.

As used herein, the terms “charge transducing” and “charge transferring” are used interchangeably.

Labeling with detectable moieties can be carried out either prior to or after conjugate formation, or prior to or after binding of the conjugate to the target nucleic acid. In preferred embodiments, a single target nucleic acid molecule is bound by several different conjugates at a given time and thus it is advisable to label such conjugates prior to nucleic acid molecule binding. If however, the detectable moiety is an antibody or a fragment thereof, then it will be possible to detect the conjugate following binding to the nucleic acid particularly if the antibody or fragment thereof is specific for the nucleic acid binding agent and each conjugate contains an immunologically distinct binding agent (so that there is no cross reaction between conjugates).

Other detectable labels include radioactive isotopes such as p³² or H³, optical or electron density markers, etc., biotin, digoxigenin, or epitope tags such as the FLAG epitope or the HA epitope, biotin, avidin and enzyme tags such as alkaline phosphatase, horseradish peroxidase, β-galactosidase, etc. Other labels include chemiluminescent substrates, chromogenic substrates, fluorophores such as fluorescein (e.g., fluorescein succinimidyl ester), TRITC, rhodamine, tetramethylrhodamine, R-phycoerythrin, Cy-3, Cy-5, Cy-7, Texas Red, Phar-Red, allophycocyanin (APC), etc. Also envisioned by the invention is the use of semiconductor nanocrystals such as quantum dots, described in U.S. Pat. No. 6,207,392 as labels. Quantum dots are commercially available from Quantum Dot Corporation. The labels (i.e., tags) may be directly linked to the DNA bases or may be secondary or tertiary units linked to modified DNA bases.

In some embodiments, the conjugates of the invention are labeled with detectable moieties that emit distinguishable signals that can all be detected by one type of detection system. For example, the detectable moieties can all be fluorescent labels or radioactive labels. In other embodiments, the conjugates are labeled with moieties that are detected using different detection systems. For example, one conjugate may be labeled with a fluorophore while another may be labeled with radioactivity.

Analysis of the nucleic acid involves detecting signals from the labels (potentially through the use of a secondary label, as the case may be), and determining the relative position of those labels relative to one another. In some instances, it may be desirable to further label the nucleic acid molecule with a standard marker that facilitates comparing the information so obtained with that from other nucleic acids analyzed. For example, the standard marker may be a backbone label, or a label that binds to a particular sequence of nucleotides (be it a unique sequence or not), or a label that binds to a particular location in the nucleic acid molecule (e.g., an origin of replication, a transcriptional promoter, a centromere, etc.).

One subset of backbone labels are nucleic acid stains that bind nucleic acids in a sequence independent manner. Examples include intercalating dyes such as phenanthridines and acridines (e.g., ethidium bromide, propidium iodide, hexidium iodide, dihydroethidium, ethidium homodimer-1 and -2, ethidium monoazide, and ACMA); minor grove binders such as indoles and imidazoles (e.g., Hoechst 33258, Hoechst 33342, Hoechst 34580 and DAPI); and miscellaneous nucleic acid stains such as acridine orange (also capable of intercalating), 7-AAD, actinomycin D, LDS751, and hydroxystilbamidine. All of the aforementioned nucleic acid stains are commercially available from suppliers such as Molecular Probes, Inc. Still other examples of nucleic acid stains include the following dyes from Molecular Probes: cyanine dyes such as SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-], TO-PRO-3, TO-PRO-5, JO-PRO-1, LOPRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -1, -20, -22, -15, -14, -25 (green), SYTO-81, -80, -82, -83, -84, -85 (orange), SYTO-64, -17, -59, -61, -62, -60, -63 (red).

In some embodiments, it is more desirable to label the nucleic acid binding agent than the tag molecule particularly if the labeling of the tag molecule negatively impacts upon the binding of the tag molecule.

The nucleic acid tag molecules and/or the nucleic acid binding agents can be labeled using antibodies or antibody fragments and their corresponding antigen or hapten binding partners. Detection of such bound antibodies and proteins or peptides is accomplished by techniques well known to those skilled in the art. Use of hapten conjugates such as digoxigenin or dinitrophenyl is also well suited herein. Antibody/antigen complexes which form in response to hapten conjugates are easily detected by linking a label to the hapten or to antibodies which recognize the hapten and then observing the site of the label. Alternatively, the antibodies can be visualized using secondary antibodies or fragments thereof that are specific for the primary antibody used. Polyclonal and monoclonal antibodies may be used. Antibody fragments include Fab, F(ab)₂, Fd and antibody fragments which include a CDR3 region. The conjugates can also be labeled using dual specificity antibodies.

In some instances, the conjugates of the invention can be further labeled with cytotoxic agents (e.g., antibiotics) or nucleic acid cleaving enzymes. In this way, the conjugates can be used for therapeutic purposes as well as for nucleic acid detection and analysis. This may be particularly useful where the tag molecule has sequence specificity to a known genetic mutation or translocation associated with a disorder or predisposition to a disorder.

The nucleic acid molecules are analyzed using linear polymer analysis systems. A linear polymer analysis system is a system that analyzes polymers in a linear manner (i.e., starting at one location on the polymer and then proceeding linearly in either direction therefrom). As a polymer is analyzed, the detectable labels attached to it are detected in either a sequential or simultaneous manner. When detected simultaneously, the signals usually form an image of the polymer, from which distances between labels can be determined. When detected sequentially, the signals are viewed in histogram (signal intensity vs. time), that can then be translated into a map, with knowledge of the velocity of the nucleic acid molecule. It is to be understood that in some embodiments, the nucleic acid molecule is attached to a solid support, while in others it is free flowing. In either case, the velocity of the nucleic acid molecule as it moves past, for example, an interaction station or a detector, will aid in determining the position of the labels, relative to each other and relative to other detectable markers that may be present on the nucleic acid molecule.

Accordingly, the linear polymer analysis systems are able to deduce not only the total amount of label on a nucleic acid molecule, but perhaps more importantly, the location of such labels. The ability to locate and position the labels allows these patterns to be superimposed on other genetic maps, in order to orient and/or identify the regions of the genome being analyzed. In preferred embodiments, the linear polymer analysis systems are capable of analyzing nucleic acid molecules individually (i.e., they are single molecule detection systems).

An example of such a system is the Gene Engine™ system described in PCT patent applications WO98/35012 and WO00/09757, published on Aug. 13, 1998, and Feb. 24, 2000, respectively, and in issued U.S. Pat. No. 6,355,420 B1, issued Mar. 12, 2002. The contents of these applications and patent, as well as those of other applications and patents, and references cited herein are incorporated by reference in their entirety. This system allows single nucleic acid molecules to be passed through an interaction station in a linear manner, whereby the nucleotides in the nucleic acid molecules are interrogated individually in order to determine whether there is a detectable label conjugated to the nucleic acid molecule. Interrogation involves exposing the nucleic acid molecule to an energy source such as optical radiation of a set wavelength. In response to the energy source exposure, the detectable label on the nucleotide (if one is present) emits a detectable signal. The mechanism for signal emission and detection will depend on the type of label sought to be detected.

Other single molecule nucleic acid analytical methods which involve elongation of DNA molecule can also be used in the methods of the invention. These include optical mapping (Schwartz, D. C. et al., Science 262(5130):110-114 (1993); Meng, X. et al., Nature Genet. 9(4):432-438 (1995); Jing, J. et al., Proc. Natl. Acad. Sci. USA 95(14):8046-8051 (1998); and Aston, C. et al., Trends Biotechnol. 17(7):297-302 (1999)) and fiber-fluorescence in situ hybridization (fiber-FISH) (Bensimon, A. et al., Science 265(5181):2096-2098 (1997)). In optical mapping, nucleic acid molecules are elongated in a fluid sample and fixed in the elongated conformation in a gel or on a surface. Restriction digestions are then performed on the elongated and fixed nucleic acid molecules. Ordered restriction maps are then generated by determining the size of the restriction fragments. In fiber-FISH, nucleic acid molecules are elongated and fixed on a surface by molecular combing. Hybridization with fluorescently labeled probe sequences allows determination of sequence landmarks on the nucleic acid molecules. Both methods require fixation of elongated molecules so that molecular lengths and/or distances between markers can be measured. Pulse field gel electrophoresis can also be used to analyze the labeled nucleic acid molecules. Pulse field gel electrophoresis is described by Schwartz, D. C. et al., Cell 37(1):67-75 (1984). Other nucleic acid analysis systems are described by Otobe, K. et al., Nucleic Acids Res. 29(22):E109 (2001), Bensimon, A. et al. in U.S. Pat. No. 6,248,537, issued Jun. 19, 2001, Herrick, J. et al., Chromosome Res. 7(6):409:423 (1999), Schwartz in U.S. Pat. No. 6,150,089 issued Nov. 21, 2000 and U.S. Pat. No. 6,294,136, issued Sep. 25, 2001. Other linear polymer analysis systems can also be used, and the invention is not intended to be limited to solely those listed herein.

The nature of such detection systems will depend upon the nature of the detectable moiety used to label the conjugate, conjugate components, and nucleic acid. The detection system can be selected from any number of detection systems known in the art. These include an electron spin resonance (ESR) detection system, a charge coupled device (CCD) detection system, a fluorescent detection system, an electrical detection system, a photographic film detection system, a chemiluminescent detection system, an enzyme detection system, an atomic force microscopy (AFM) detection system, a scanning tunneling microscopy (STM) detection system, an optical detection system, a nuclear magnetic resonance (NMR) detection system, a near field detection system, and a total internal reflection (TIR) detection system, many of which are electromagnetic detection systems.

The binding pattern of the conjugates of the invention to target nucleic acids can be used to derive sequence information about the targets such as DNA physical maps. As mentioned above, the length of the tag molecule (and thus its complementary sequence) controls to some extent the resolution of such information. For example, if the tag molecule is long, then the resolution will be low. The shorter the tag molecule, the higher the potential resolution will be, provided that contiguously positioned conjugates can be discerned from each other. That is, the contiguously positioned conjugates should be spaced at a distance that is greater than the resolution limit of the detection system used.

### Equivalents

It should be understood that the preceding is merely a detailed description of certain embodiments. It therefore should be apparent to those of ordinary skill in the art that various modifications and equivalents can be made without departing from the spirit and scope of the invention, and with no more than routine experimentation. It is intended to encompass all such modifications and equivalents within the scope of the appended claims.

All references, patents and patent applications that are recited in this application are incorporated by reference herein in their entirety.

I claim:

1. A method for analyzing a polymer comprising
contacting the polymer with a conjugate comprising a nucleic acid tag molecule and a nucleic acid binding agent,
allowing the nucleic acid binding agent to bind to the polymer non-specifically, and
allowing the nucleic acid tag molecule to bind specifically to the polymer, and
determining a pattern of binding of the conjugate to the polymer.

2. The method of claim 1, further comprising allowing the nucleic acid binding agent to translocate along the polymer.

3. The method of claim 1, wherein the nucleic acid binding agent binds to the polymer non-specifically.

4. The method of claim 1, wherein the polymer is a nucleic acid molecule.

5. The method of claim 1, wherein the polymer is DNA or RNA.

6. The method of claim 1, wherein the nucleic acid tag molecule is selected from the group consisting of a peptide nucleic acid (PNA), a locked nucleic acid (LNA), a DNA, an RNA, a bisPNA clamp, a pseudocomplementary PNA, and a LNA-DNA co-polymer.

7. The method of claim 1, wherein the nucleic acid tag molecule is 5-50 residues in length.

8. The method of claim 1, wherein the nucleic acid tag molecule and the nucleic acid binding agent are covalently linked to each other.

9. The method of claim 1, wherein the nucleic acid tag molecule and the nucleic acid binding agent are conjugated using a linker molecule.

10. The method of claim 1, wherein the nucleic acid binding agent is an enzyme.

11. The method of claim 10, wherein the enzyme is selected from the group consisting of a DNA polymerase, an RNA polymerase, a DNA repair enzyme, a helicase, a nuclease, and a ligase.

12. The method of claim 10, wherein the enzyme lacks the ability to modify the nucleic acid tag molecule or the polymer.

13. The method of claim 1, wherein the nucleic acid tag molecule is labeled with a detectable moiety.

14. The method of claim 1, wherein the nucleic acid binding agent is labeled with a detectable moiety.

15. The method of claim 1, wherein the nucleic acid tag molecule is labeled with a first detectable moiety, and the nucleic acid binding agent is labeled with a second detectable moiety.

16. The method of claim 1, wherein the polymer is labeled with a detectable moiety.

17. The method of claim 16, wherein the detectable moiety is a backbone specific label.

18. The method of claim 1, wherein the nucleic acid binding agent is not itself a detectable moiety.

19. The method of claim 1, wherein the pattern of binding of the conjugate to the polymer is determined using a linear polymer analysis system.

20. The method of claim 19, wherein the linear polymer analysis system comprises exposing the polymer to a station to produce a signal arising from the binding of the conjugate to the polymer, and detecting the signal using a detection system.

21. The method of claim 1, wherein the pattern of binding of the conjugate to the polymer is determined using fluorescence in situ hybridization (FISH).

22. The method of claim 13, 14, or 15, wherein the detectable moiety is selected from the group consisting of an electron spin resonance molecule, a fluorescent molecule, a chemiluminescent molecule, a radioisotope, an enzyme substrate, a biotin molecule, an avidin molecule, an electrical charged transferring molecule, a semiconductor nanocrystal, a semiconductor nanoparticle, a colloid gold nanocrystal, a ligand, a microbead, a magnetic bead, a paramagnetic particle, a quantum dot, a chromogenic substrate, an affinity molecule, a protein, a peptide, a nucleic acid, a carbohydrate, an antigen, a hapten, an antibody, an antibody fragment, and a lipid.

23. The method of claim 22, wherein the detectable moiety is detected using a detection system selected from the group consisting of an electron spin resonance detection system, a charge coupled device (CCD) detection system, a fluorescent detection system, an electrical detection system, a photographic film detection system, a chemiluminescent detection system, an enzyme detection system, an atomic force microscopy (AFM) detection system, a scanning tunneling microscopy (STM) detection system, an optical detection system, a nuclear magnetic resonance (NMR) detection system, a near field detection system, and a total internal reflection (TIR) detection system.

24. The method of claim 1, wherein the polymer is a non in vitro amplified nucleic acid molecule.

25. The method of claim 1, wherein the nucleic acid tag molecule is not an antisense molecule.

26. The method of claim 1, wherein the nucleic acid tag molecule does not hybridize to bacterial or viral specific sequences.

27. The method of claim 1, wherein the nucleic acid tag molecule is labeled with an agent.

28. The method of claim 27, wherein the agent is capable of cleaving a nucleic acid molecule.

29. The method of claim 28, wherein the agent is a photocleaving agent.

30. The method of claim 27, wherein the agent is able to modify a nucleic acid molecule.

31. The method of claim 1, wherein the nucleic acid binding agent is detected indirectly.

32. The method of claim 31, wherein the nucleic acid binding agent is detected indirectly using an antibody or an antibody fragment specific for the nucleic acid binding agent.

33. The system of claim 19, wherein the linear polymer analysis system is a single polymer analysis system.

34. The system of claim 1, wherein the pattern of binding of the conjugate to the polymer is determined using a method selected from the group consisting of Gene Engine™, optical mapping, and DNA combing.

35. A system for optically analyzing a polymer comprising:
an optical source for emitting optical radiation;
an interaction station for receiving the optical radiation and for receiving a polymer that is exposed to the optical radiation to produce detectable signals; and
a processor constructed and arranged to analyze the polymer based on the detected radiation including the signals,
wherein the polymer is bound to a conjugate comprising a nucleic acid tag molecule and a nucleic acid binding agent.

36. The system of claim 35, wherein the polymer is a nucleic acid molecule.

37. The system of claim 35, wherein the polymer is DNA or RNA.

38. The system of claim 35, wherein the nucleic acid tag molecule of the conjugate is selected from the group consisting of a peptide nucleic acid (PNA), a locked nucleic acid (LNA), a DNA, an RNA, a bisPNA clamp, a pseudocomplementary PNA, and a LNA-DNA co-polymer.

39. The system of claim 35, wherein the nucleic acid tag molecule is 5-50 residues in length.

40. The system of claim 35, wherein the nucleic acid tag molecule and the nucleic acid binding agent are covalently conjugated to each other.

41. The system of claim 35, wherein the nucleic acid tag molecule and the nucleic acid binding agent are conjugated to each other using a linker molecule.

42. The system of claim 35, wherein the nucleic acid binding agent is an enzyme.

43. The system of claim 42, wherein the enzyme is selected from the group consisting of a DNA polymerase, an RNA polymerase, a DNA repair enzyme, a helicase, a nuclease, and a ligase.

44. The system of claim 42, wherein the enzyme lacks the ability to modify the nucleic acid tag molecule or the polymer.

45. The system of claim 35, wherein the nucleic acid tag molecule is labeled with a detectable moiety.

46. The system of claim 35, wherein the nucleic acid binding agent is labeled with a detectable moiety.

47. The system of claim 35, wherein the nucleic acid tag molecule is labeled with a first detectable moiety, and the nucleic acid binding agent is labeled with a second detectable moiety.

48. The system of claim 35, wherein the polymer is labeled with a detectable moiety.

49. The system of claim 48, wherein the detectable label is a backbone specific label.

50. The system of claim 45, 46, or 47, wherein the detectable moiety is selected from the group consisting of an electron spin resonance molecule, a fluorescent molecule, a chemiluminescent molecule, a radioisotope, an enzyme substrate, a biotin molecule, an avidin molecule, an electrical charged transferring molecule, a semiconductor nanocrystal, a semiconductor nanoparticle, a colloid gold nanocrystal, a ligand, a microbead, a magnetic bead, a paramagnetic particle, a quantum dot, a chromogenic substrate, an affinity molecule, a protein, a peptide, a carbohydrate, an antibody, an antibody fragment, an antigen, a hapten, and a lipid.

51. The system of claim 50, wherein the detectable moiety is detected using a detection system selected from the group consisting of a charge coupled device (CCD) detection system, an electron spin resonance detection system, a fluorescent detection system, an electrical detection system, a photographic film detection system, a chemiluminescent detection system, an enzyme detection system, an atomic force microscopy (AFM) detection system, a scanning tunneling microscopy (STM) detection system, an optical detection system, a nuclear magnetic resonance (NMR) detection system, a near field detection system, and a total internal reflection (TIR) detection system.

52. The system of claim 35, wherein the polymer is a non in vitro amplified nucleic acid molecule.

53. The system of claim 35, wherein the interaction station includes a slit having a slit width in a range of 1 nm to 500 nm and producing a localized radiation spot.

54. The system of claim 53, wherein the slit width is in a range of 10 nm to 100 nm.

55. The system of claim 53, further comprising a microchannel arranged with the slit to produce the localized radiation spot, the microchannel being constructed to receive and advance the polymer through the localized radiation spot.

56. The system of claim 53, further comprising a polarizer, wherein the optical source includes a laser constructed to emit a beam of radiation and the polarizer is arranged to polarize the beam prior to reaching the slit.

57. The system of claim 56, wherein the polarizer is arranged to polarize the beam parallel to the width of the slit.

58. The system of claim 35, further comprising a microchannel arranged to produce a localized radiation spot, the microchannel being constructed to receive and advance the polymer through the localized radiation spot.

59. The system of claim 35, further comprising a polarizer, wherein the optical source includes a laser constructed to emit a beam of radiation and the polarizer is arranged to polarize the beam.

60. The system of claim 35, wherein the optical source is a light source integrated on a chip.

61. The system of claim 35, wherein the conjugate of the nucleic acid tag molecule and the nucleic acid binding agent is specifically bound to the polymer.

62. The system of claim 35, wherein the nucleic acid binding agent is bound non-specifically to the polymer.

63. The system of claim 35, wherein the nucleic acid binding agent is detected indirectly.

64. The system of claim 63, wherein the nucleic acid binding agent is detected indirectly using an antibody or an antibody fragment specific for the nucleic acid binding agent.

65. The system of claim 51 wherein the detection system is incorporated into a linear polymer analysis system.

66. The system of claim 65, wherein the linear polymer analysis system is a single polymer analysis system.

67. The system of claim 35, wherein the polymer is analyzed using a method selected from the group consisting of Gene Engine™, optical mapping, and DNA combing.

68. A method for analyzing a polymer comprising:
generating optical radiation of a known wavelength to produce a localized radiation spot;
passing a polymer through a microchannel;
irradiating the polymer at the localized radiation spot;
sequentially detecting radiation resulting from interaction of the polymer with the optical radiation at the localized radiation spot; and
analyzing the polymer based on the detected radiation,
wherein the polymer is bound to a conjugate of a nucleic acid tag molecule and a nucleic acid binding agent.

69. The method of claim 68, wherein the polymer is a nucleic acid molecule.

70. The method of claim 69, further comprising employing an electric field to pass the nucleic acid molecule through the microchannel.

71. The method of claim 69, wherein the detecting includes collecting the signals over time while the nucleic acid molecule is passing through the microchannel.

72. The method of claim 68, wherein the nucleic acid tag molecule of the conjugate binds specifically to the polymer and the nucleic acid binding agent binds non-specifically to the polymer.

73. The method of claim 69, wherein the nucleic acid molecule is DNA or RNA.

74. The method of claim 68, wherein the nucleic acid molecule of the conjugate is selected from the group consisting of a peptide nucleic acid (PNA), a locked nucleic acid (LNA), a DNA, an RNA, a bisPNA clamp, a pseudocomplementary PNA, and a LNA-DNA co-polymer.

75. The method of claim 69, wherein the nucleic acid molecule is 5-50 residues in length.

76. The method of claim 68, wherein the nucleic acid tag molecule and the nucleic acid binding agent are covalently conjugated to each other.

77. The method of claim 68, wherein the nucleic acid molecule and the nucleic acid binding agent are conjugated to each other using a linker molecule.

78. The method of claim 68, wherein the nucleic acid binding agent is an enzyme.

79. The method of claim 78, wherein the enzyme is selected from the group consisting of a DNA polymerase, an RNA polymerase, a DNA repair enzyme, a helicase, a nuclease, and a ligase.

80. The method of claim 78, wherein the enzyme lacks the ability to modify a nucleic acid molecule.

81. The method of claim 68, wherein the nucleic acid tag molecule is labeled with a detectable moiety.

82. The method of claim 68, wherein the nucleic acid binding agent is labeled with a detectable moiety.

83. The method of claim 68, wherein the nucleic acid molecule is labeled with a first detectable moiety, and the nucleic acid binding agent is labeled with a second detectable moiety.

84. The method of claim 68, wherein the polymer is labeled with a detectable moiety.

85. The method of claim 84, wherein the detectable moiety is a backbone specific label.

86 The method of claim 81, 82, or 83, wherein the detectable moiety is selected from the group consisting of an electron spin resonance molecule, a fluorescent molecule, a chemiluminescent molecule, a radioisotope, an enzyme substrate, a biotin molecule, an avidin molecule, an electrical charged transferring molecule, a semiconductor nanocrystal, a semiconductor nanoparticle, a colloid gold nanocrystal, a ligand, a microbead, a magnetic bead, a paramagnetic particle, a quantum dot, a chromogenic substrate, an affinity molecule, a protein, a peptide, nucleic acid, a carbohydrate, an antigen, a hapten, an antibody, an antibody fragment, and a lipid.

87. The method of claim 86, wherein the detectable moiety is detected using a detection system selected from the group consisting of an electron spin resonance detection system, a charge coupled device detection system, a fluorescent detection system, an electrical detection system, a photographic film detection system, a chemiluminescent detection system, an enzyme detection system, an atomic force microscopy (AFM) detection system, a scanning tunneling microscopy (STM) detection system, an optical detection system, a nuclear magnetic resonance (NMR) detection system, a near field detection system, and a total internal reflection (TIR) detection system.

88. The method of claim 69, wherein the nucleic acid is a non in vitro amplified nucleic acid molecule.

89. The method of claim 68, wherein the nucleic acid binding agent is detected indirectly.

90. The method of claim 89, wherein the nucleic acid binding agent is detected indirectly using an antibody or an antibody fragment specific for the nucleic acid binding agent.

91. A method for analyzing a nucleic acid molecule, comprising:
exposing a nucleic acid molecule to a conjugate of a nucleic acid tag molecule and a nucleic acid binding enzyme,
allowing the nucleic acid binding enzyme to bind to the nucleic acid molecule,
allowing the nucleic acid tag molecule to bind to the nucleic acid molecule in a sequence-specific manner, and
determining a pattern of binding of the conjugate to the nucleic acid molecule.

92. The method of claim 91, wherein the nucleic acid binding enzyme binds to the nucleic acid molecule non-specifically.

93. The method of claim 91, wherein the nucleic acid molecule is DNA or RNA.

94. The method of claim 91, wherein the nucleic acid tag molecule is selected from the group consisting of a peptide nucleic acid (PNA), a locked nucleic acid (LNA), a DNA, an RNA, a bisPNA, a pseudocomplementary PNA, and a LNA-DNA co-polymer.

95. The method of claim 91, wherein the nucleic acid tag molecule is 5-50 residues in length.

96. The method of claim 91, wherein the nucleic acid tag molecule and the nucleic acid binding enzyme are covalently linked to each other.

97. The method of claim 91, wherein the nucleic acid tag molecule and the nucleic acid binding enzyme are conjugated to each other using a linker.

98. The method of claim 91, wherein the nucleic acid binding enzyme is selected from the group consisting of a DNA polymerase, an RNA polymerase, a DNA repair enzyme, a helicase, a nuclease, and a ligase.

99. The method of claim 98, wherein the enzyme nucleic acid binding lacks the ability to modify the nucleic acid molecule or the tag molecule.

100. The method of claim 91, wherein the nucleic acid tag molecule is labeled with a detectable moiety.

101. The method of claim 91, wherein the nucleic acid binding enzyme is labeled with a detectable moiety.

102. The method of claim 91, wherein the nucleic acid tag molecule is labeled with a first detectable moiety, and the nucleic acid binding enzyme is labeled with a second detectable moiety.

103. The method of claim 91, wherein the nucleic acid molecule is labeled with a detectable moiety.

104. The method of claim 91, wherein the nucleic acid molecule is labeled with a backbone specific label.

105. The method of claim 91, wherein the pattern of binding of the conjugate to the nucleic acid molecule is determined using a linear nucleic acid analysis system.

106. The method of claim 105, wherein the linear nucleic acid analysis system comprises exposing the polymer to a station to produce a signal arising from the binding of the conjugate to the polymer, and detecting the signal using a detection system.

107. The method of claim 100, 101, or 102, wherein the detectable moiety is selected from the group consisting of an electron spin resonance molecule, a fluorescent molecule, a chemiluminescent molecule, a radioisotope, an enzyme substrate, a biotin molecule, an avidin molecule, an electrical charged transferring molecule, a semiconductor nanocrystal, a semiconductor nanoparticle, a colloid gold nanocrystal, a ligand, a microbead, a magnetic bead, a paramagnetic bead, a quantum dot, a chromogenic substrate, an affinity molecule, a protein, a peptide, a nucleic acid, a hapten, an antigen, an antibody, an antibody fragment, a carbohydrate, and a lipid.

108. The method of claim 107, wherein the detectable moiety is detected using a detection system selected from the group consisting of an electron spin resonance detection system, a charge coupled device detection system, a fluorescent detection system, an electrical detection system, a photographic film detection system, a chemiluminescent detection system, an enzyme detection system, an atomic force microscopy (AFM) detection system, a scanning tunneling microscopy (STM) detection system, an optical detection system, a nuclear magnetic resonance (NMR) detection system, a near field detection system, and a total internal reflection (TIR) system.

109. The method of claim 91, wherein the nucleic acid molecule is a non in vitro amplified nucleic acid molecule.

110. The method of claim 91, wherein the nucleic acid binding agent is detected indirectly.

111. The method of claim 110, wherein the nucleic acid binding agent is detected indirectly using an antibody or an antibody fragment specific for the nucleic acid binding agent.

112. A composition comprising
a conjugate of a nucleic acid tag molecule and a nucleic acid binding enzyme,
wherein a detectable moiety is present on the nucleic acid binding enzyme.

113. A composition comprising
a conjugate of a nucleic acid tag molecule and a nucleic acid binding enzyme,
wherein a detectable moiety is present on the nucleic acid tag molecule and wherein the nucleic acid binding enzyme is not the detectable moiety.

114. The composition of claim 112 or 113, wherein the nucleic acid tag molecule and the nucleic acid binding agent are covalently linked to each other.

115. The composition of claim 112 or 113, wherein the nucleic acid tag molecule and the nucleic acid binding agent are linked to each other using a linker molecule.

116. The composition of claim 112 or 113, wherein the nucleic acid tag molecule is selected from the group consisting of a peptide nucleic acid (PNA), a locked nucleic acid (LNA), a DNA, an RNA, a bisPNA clamp, a pseudocomplementary PNA, and a LNA-DNA co-polymer.

117. The composition of claim 112 or 113, wherein the nucleic acid binding enzyme is selected from the group consisting of a DNA polymerase, an RNA polymerase, a DNA repair enzyme, a helicase, a nuclease, and a ligase.

118. The composition of claim 112 or 113, wherein the nucleic acid binding enzyme lacks the ability to modify a nucleic acid molecule.

119. The composition of claim 112, wherein the nucleic acid tag molecule is labeled with a second detectable moiety.

120. The composition of claim 113, wherein the nucleic acid binding enzyme is labeled with a second detectable moiety.

121. The composition of claim 112, 113, 119 or 120, wherein the detectable moiety is selected from the group consisting of an electron spin resonance molecule, a fluorescent molecule, a chemiluminescent molecule, a radioisotope, an enzyme substrate, a biotin molecule, an avidin molecule, an electrical charged transferring molecule, a semiconductor nanocrystal, a semiconductor nanoparticle, a ligand, a microbead, a magnetic bead, a paramagnetic molecule, a quantum dot, a chromogenic substrate, an affinity molecule, a protein, a peptide, nucleic acid, a carbohydrate, a hapten, an antigen, an antibody, an antibody fragment, and a lipid.

122. The composition of claim 121, wherein the detectable moiety is detected using a detection system selected from the group consisting of an electric spin resonance detection system, a charge coupled device detection system, a fluorescent detection system, an electrical detection system, a photographic film detection system, a chemiluminescent detection system, an enzyme detection system, an atomic force microscopy (AFM) detection system, a scanning tunneling microscopy (STM) detection system, an optical detection system, a nuclear magnetic resonance (NMR) detection system, a near field detection system, and a total internal reflection (TIR) system.

123. The method of claim 112, wherein the nucleic acid binding agent is detected indirectly.

124. The method of claim 123, wherein the nucleic acid binding agent is detected indirectly using an antibody or an antibody fragment specific for the nucleic acid binding agent.

125. A method for analyzing a polymer comprising
contacting the polymer with a conjugate comprising a nucleic acid tag molecule and a nucleic acid binding agent,
allowing the nucleic acid binding agent to bind to the polymer, and
allowing the nucleic acid tag molecule to bind specifically to the polymer,
wherein the nucleic acid binding agent is selected from the group consisting of a DNA repair enzyme, a helicase, a nuclease, and a ligase.

126. A method for labeling a polymer comprising
contacting the polymer with a conjugate comprising a nucleic acid tag molecule and a nucleic acid binding agent,
allowing the nucleic acid binding agent to bind to and translocate along the polymer, and
allowing the nucleic acid tag molecule to bind specifically to the polymer.

127. The method of claim 126, wherein the nucleic acid binding agent binds to the polymer non-specifically.

128. The method of claim 126, further comprising determining a pattern of binding of the conjugate to the polymer.
